(19) United States
(12) Patent Application Publication
(10) Pub. No.: US 2012/0241393 A1
(43) Pub. Date: Sep. 27, 2012

(54) SUPPORT SYSTEM FOR SCANNERS

(75) Inventors: Ernst Roth, Langenthal (CH); Walter Zulauf, Ursenbach (CH)

(73) Assignee: GUDEL GROUP AG, Langenthal (CH)

(21) Appl. No.: 13/496,511

(22) PCT Filed: Sep. 16, 2010
(86) PCT No.: PCT/CH2010/000223

§ 371 Date: May 29, 2012

(30) Foreign Application Priority Data
Sep. 18, 2009 (EP) 09 405 160.4

(51) Int. Cl. A61B 19/00; H05K 7/18

(52) U.S. Cl. 211/26

(57) ABSTRACT

A support system for a scanner comprises a frame and a drum. The drum is made of tempered steel and is mounted on the frame by a roller system. The roller system comprises rollers of hardened steel, or having roller surfaces of hardened steel, that allow the drum to rotate about a rotationally symmetrical axis of the drum. A radiation source and one or more detectors can be secured to the drum for a scanner. Because that the drum is made of tempered steel and the rollers have surfaces of hardened steel, the drum can rotate at a high speed without increasing vibrations, thus allowing examination times of the scanner to be short and preventing vibration-related background signals. Also, noise occurring during use of the scanner is reduced.

### TECHNICAL FIELD

The invention relates to a support system for a scanner, comprising a frame and a drum, wherein mounting of the drum on the frame allows a rotation of the drum about the axis of rotational symmetry thereof and wherein a scanner system can be attached to the drum.

### PRIOR ART

Support systems, of the type mentioned above, for scanners are known. In particular, the scanner system can be an X-ray radiation source and one or more corresponding detectors. By way of example, DE 196 29 931 C2 (Siemens) describes an X-ray computed tomography scanner with an annular gantry that is rotatably mounted in a frame which has rollers for the radial and axial guidance of the gantry. In this case, the axles of the rollers are offset with respect to one another by 90° and run on the outer or lateral faces of rails that enclose the gantry along the circumference thereof. The rails are connected to the gantry with a rubber layer disposed therebetween.

DE 195 81 512 C2 (Analogic) relates to an X-ray beam tomography system with a pivotable frame, on which a rotatable drum is arranged. Functional elements of the system (at least the X-ray source) are attached to the drum. The drum is mounted on carriages that comprise tandem rollers which are rotatable about an axis that is parallel to the rotational axis of the drum. The bearing surfaces of the rollers and the carriage are manufactured from an elastic material and thus dampen a transmission of vibrations between the frame and the drum, and absorb cyclical temperature variations of the drum. The peripheral, annular component of the drum is formed by a rolled extruded part made of a light material such as e.g. aluminum or an Mg—Al alloy. The component forms a uniform, circular band with edge lips protruding in the transverse direction, said edge lips extending outward in the radial direction. In addition to the carriage, the device has at least three pairs of elastic rollers, which have been provided with tires, which are arranged on the frame for rotation along radial lines. These rollers contact the lateral edges of the edge lips of the drum and hence guide the latter in the axial direction. The drum is driven by a motor which is coupled to one of the tandem rollers.

U.S. Pat. No. 7,010,081 B2 (Philips) shows a CT scanner with a rotating gantry and a stationary gantry, wherein the rotating gantry has an encircling mounting rail, which has raceways tilted toward one another for axial and radial mounting. Conical rollers arranged on the stationary gantry interact in pairs with the raceways of the mounting rail. A drive motor is coupled to one of the rollers for driving the rotating gantry. The roller bearings consist of a metallic core, more particularly steel, and a tire-like polymer coating, more particularly made of polyurethane, which creates cushion-like damping while at the same time having a rigid mounting.

It was found that the rotational movement of the gantry cannot be controlled precisely in such devices and that undesired flexing of the gantry may occur.

U.S. Pat. No. 5,109,397 (Analogic) shows a computed tomography scanner with an annular gantry. The latter is attached to a vertical support frame such that it can pivot about a horizontal axis, wherein this support frame can be linearly displaced along rails relative to an instrument base. A lever whose length can be adjusted is attached between the support frame and the gantry and enables the pivoting of the gantry. The gantry comprises an external annular frame and a rotatable inner part that is attached to the inside of the frame by antifriction bearings.

This solution has a complicated design.

U.S. Pat. No. 6,188,743 B1 (Analogic) relates to a computed tomography scanner with an annular disk, the circumferential edge of which is provided with grooves so that the disk can serve as a belt pulley which can be driven by a drive motor via a tensioned belt. The disk is mounted on a stationary frame, either in the region of its center of gravity via spacers or on the circumference by conventional four-wire mounts. The disk can be manufactured from a light, stiff material such as e.g. aluminum or a magnesium-aluminum alloy and is preferably produced by precision casting and is subsequently hardened and subjected to finishing.

Conventional wire bearings have soldering points of the wires, which lead to vibrations in the case of a high rotational speed of the disk. These vibrations cause a background signal in the tomographic recordings.

### DESCRIPTION OF THE INVENTION

It is an object of the invention to create a support system that is part of the technical field mentioned at the outset, which enables short examination times but nevertheless has a cost-effective design.

The solution to the object is defined by the features of claim 1. According to the invention, the drum substantially consists of heated and tempered steel and/or has bearing surfaces made of hardened steel and a roller system with rollers is attached to the frame, by means of which roller system the drum can be rotated about the axis of rotational symmetry of the drum. The rollers consist of hardened steel or are at least equipped with bearing surfaces made of hardened steel.

As a result of the drum being produced from heated and tempered steel and as a result of the rollers, which have at least bearing surfaces made of hardened steel, it is possible to achieve increased rotational speeds of the drum, without increased vibrations of the drum occurring at the same time. The rotational speeds achieved in this manner enable short examination times of the scanner because vibration-dependent background signals are avoided. Moreover, disadvantageous noise-development during the examination, which would decrease the comfort of the users of the scanner, is avoided.

The rollers particularly preferably consist of ball-bearing steel and are hardened through.

Mounting the drum on a roller system so that increased rotational speeds of the drum are made possible can be brought about in a simple and cost-effective manner.

Therefore the support system, according to the invention, for scanners is particularly suitable for applications in which, in a cost-effective manner, a short examination time is desired. In addition to computed tomography in the medical field, this specifically also is the case in baggage handling at airports or at customs.

The drum produced from heated and tempered steel is advantageously produced without joints, more particularly rolled or forged without joints. This prevents vibrations that would be created if one of the rollers passes over a joint in the drum. This enables a further increase in the rotational speed of the drum, without an undesired increase in the vibrations.

On the lateral face, the drum preferably comprises a rail (or bearing surface) on which the rollers of the roller system run. Together, this rail and the drum are advantageously formed integrally from the same material. Moreover, the rail preferably has induction hardened bearing surfaces.

In a preferred embodiment, the weight of the drum is borne by at least two roller pairs, which are each arranged moveably relative to the frame along a track oriented tangentially with respect to the drum (and perpendicularly to the drum rotational axis). The moveable arrangement enables a self-regulating, uniform distribution of the weight of the drum on the roller pairs. Moreover, small changes in size and shape of the drum due to temperature can be compensated for. A very small tangential displacement path of the rollers of less than e.g. 0.5% of the drum diameter has already proven to be sufficient. It is advantageous for the rollers to be free and movable along the track with as little friction as possible; however, alternatively, it is also possible to provide e.g. springs, which bring the rollers into a predetermined zero position if there are no additional forces.

Instead of roller pairs, provision can be made for spaced apart and fixedly arranged individual rollers, with the weight of the drum being borne by these.

It is preferable for two of the rollers to be attached to a roller support in each case, wherein the roller support is mounted on a linear guide attached to the frame in order to enable the tangential mobility. By mounting the drum on respectively two tangentially spaced apart rollers, the forces exerted by the drum on the roller pairs in substantially a radial direction are transferred in an optimum manner onto the roller support and, from the latter, onto the linear guide. Moreover, a damping element is particularly preferably arranged between the roller support and the linear guide. This damping element does not provide an additional movement possibility for the rollers but serves to minimize the transmission of vibrations onto the frame. As a result, the noise development can be greatly reduced. By way of example, the damping element can be embodied as a plastic coating on the linear guide.

In a further embodiment, the weight of the drum is borne by a plurality of roller pairs, which are each mounted on a rocker. Here, both the rotational axes of the rockers and the rotary axles of the rollers are oriented parallel to the axis of rotational symmetry of the drum. The rotational axis of the rockers is preferably on the mirror-symmetric axis between the two rollers in each case, said axis running perpendicular to the connecting line between the two rollers. This mounting is advantageous in that there is a self-regulating, uniform distribution of the weight of the drum on the rollers mounted on the rockers. Additionally, an advantage of mounting like this is that small changes in size and shape of the drum due to temperature are compensated for. Since this mounting of the drum adapts perpendicularly to the axis of rotational symmetry of the drum without, in doing so, providing the drum with a translational degree of freedom perpendicular to the axis of rotational symmetry of the drum, this also suppresses vibrations that may be created at high rotational speed of the drum due to a non-correspondence between the center of gravity of the drum and the rotational axis of the drum.

As an alternative to this mounting, mounting on only two rollers or mounting of the drum on a plurality of eccentrically mounted individual rollers is also feasible.

It is preferable for one or more rollers which do not bear the weight of the drum to be mounted eccentrically or to be under resilient pretension. In the following text, eccentric mounting is understood to mean eccentric mounting of the roller axle, with the eccentric being set once during assembly and remaining fixed thereafter. Resilient pretension is achieved by a spring element that places the roller onto the drum circumference. The only function of these rollers is to ensure play-free mounting of the drum. Since the rollers either are produced entirely from hardened steel or have at least one bearing surface made of hardened steel, eccentric mounting of the corresponding rollers or a resilient pretension is the best solution for this.

As an alternative to this, use can also be made of one or more rollers that consist of soft material. Such rollers adapt to the size and shape of the drum without needing to be mounted eccentrically for this purpose.

The drum is preferably held in the frame by guide rollers attached to the frame. To this end, the guide rollers run on both sides of the rail, with the axles of said guide rollers being aligned radially with respect to the drum. These guide rollers consist entirely of hardened steel (more particularly ball-bearing steel) or are equipped with at least one bearing surface made of hardened steel.

Compared to a likewise feasible arrangement in which the guide rollers run on the end faces of the drum, the guide-roller arrangement of the invention is advantageous in that the instruments and components of the scanner can also be attached to the end faces of the drum.

Compared to a further feasible arrangement of the rollers and the guide rollers on more than one rail on the lateral face of the drum, this embodiment of the invention is furthermore advantageous in that space is saved on the lateral face of the drum, and hence the scanner can be built in a space-saving manner.

It is preferable for the drum to be mounted without play by virtue of the guide rollers on at least one side of the rail being mounted eccentrically or being under resilient pretension. This enables play-free guidance of the drum, and hence quiet and uniform running with little mechanical load.

If the drum has obliquely leaning bearing surfaces, e.g. two areas respectively oriented at an angle of 45° with respect to the rotational axis, which thus include an angle of 90°, then the rollers can simultaneously also assume the guidance function for the drum; separate guide rollers become superfluous. In the case of such rollers with a guidance function, the bearing surfaces are advantageously ground like a ball such that differential slip is prevented in the region of the running contact. Rollers arranged at the top (which primarily have a guidance function) advantageously are eccentrically mounted or under resilient pretension.

The rotational movement of the drum is preferably driven by a driving belt that runs around the drum. Here, this can be any type of driving belt, e.g. a V-belt, a flat belt or a toothed belt, and the driving belt can run in a groove in the lateral face of the drum or else directly on the lateral face of the drum. The lateral face of a drum, which is provided with a rail, preferably has an integrated belt pulley next to the rail; in particular, this region of the lateral face has an appropriate profile.

The advantage of the drive via a driving belt is that there is good force transmission onto the drum. Alternative drive solutions, such as e.g. the drive by means of a driving roller or a drive roller require slip-free contact between the rail and the driving roller. Such a drive can only be achieved by a special bearing surface of the driving roller and/or by strong pressure of the driving roller against the drum. The former would have a negative influence on the rotational behavior of the drum while the latter would cause great energy loss as a result of friction.

The motor for the driving belt is preferably attached to the frame. The advantage of this is that neither the control nor the power supply for the motor needs to be routed from the frame to the drum.

The driving belt is preferably tensioned by means of a tension roller that is attached to the frame. This tension roller is advantageous in that the driving belt can be tensioned with a desired force even during operation of the support system and in that the driving belt can easily be replaced in the case of repairs.

A slip ring is preferably attached to the external side of the drum and establishes the required electrical contacts to the instruments and components that are attached to the drum, with a brush attached to the frame establishing the contacts to the slip ring. As an alternative to this, it is also possible for the required electrical contacts to be attached to the drum and the slip ring to be fixedly attached to the frame.

The contact to the slip ring on the external side of the drum preferably ensures the precise positioning of the drum.

The advantage of this type of positioning is that, unlike determining the position of the drive motor, it takes place directly on the drum and hence directly establishes the position thereof.

The instruments and components are preferably attached to holes in the drum.

This type of attaching instruments and components is advantageous in that, according to the invention, a standard embodiment of the support system can be used for different scanners and in that such a scanner can easily be upgraded with additional or newer instruments and components.

Furthermore, this type of attachment saves weight because there is no need for heavy intermediate pieces.

An alternative option for attaching the instruments and components to the drum consists of attaching a disk to the drum, with the instruments and components being fixedly mounted on said disk. In addition to the disk, it is also possible for further elements to be attached to the drum, e.g. an inner ring that interacts with the driving belt. Hence, the drum as it were acts as a raceway, onto which the further elements are added.

Further advantageous embodiments and feature combinations of the invention emerge from the following detailed description and the totality of the patent claims.

### BRIEF DESCRIPTION OF THE DRAWINGS

In the drawings used to explain the exemplary embodiment:

FIG. 1 shows an oblique view of a first support system, according to the invention, for a scanner;

FIG. 2 shows a frontal view of the support system according to the invention,

FIG. 3 shows a further oblique view of the support system according to the invention;

FIG. 4 shows an oblique view of a second support system, according to the invention, for a scanner;

FIG. 5 shows a detailed view from a first side of an installed roller support with two rollers;

FIG. 6 shows a detailed view from a second side of the roller support;

FIG. 7 shows an oblique view of the roller support;

FIG. 8 shows a cross section through the roller support;

FIG. 9 shows an oblique view of a third support system, according to the invention, for a scanner;

FIG. 10 shows a detailed view of an upper guide unit; and

FIG. 11 shows a detailed view of a lower guide unit. In principle, equivalent parts in the figures have been denoted by the same reference signs.

### WAYS OF IMPLEMENTING THE INVENTION

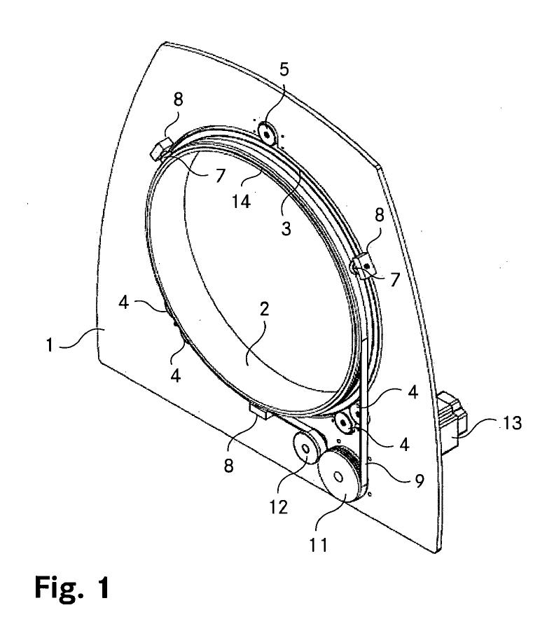

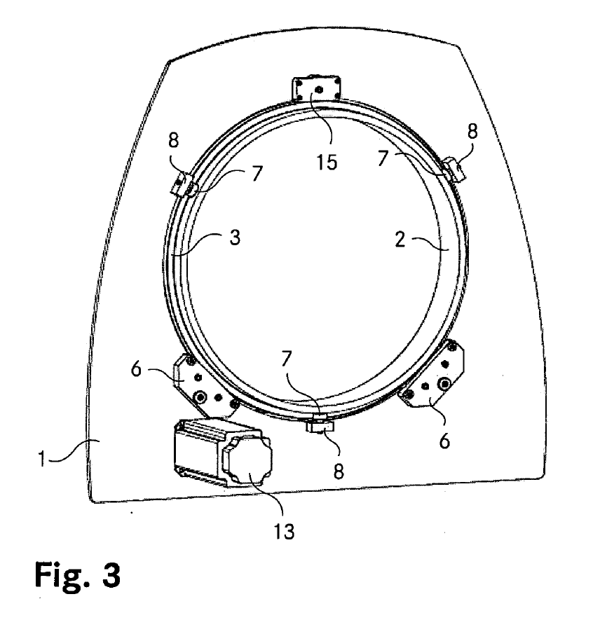

FIG. 1 shows an oblique view of a support system, according to the invention, for scanners. The illustrated support system comprises a frame 1, which consists of a steel plate and which has been attached standing up in a stand (not illustrated). Situated within this frame 1 there is an opening, in which a substantially hollow-cylindrical drum 2 has space, with the axis of rotational symmetry thereof being perpendicular with respect to the plane of the steel plate of the frame 1. The diameter of this drum 2 is approximately eight times greater than the length of the drum 2. The drum 2 is forged without joints from heated and tempered steel C45 (pursuant to DIN EN 10027-1). A rail 3 with a rectangular cross section, which runs in the center of the lateral face of the drum 2, encompasses the drum 2 in an annular manner. Together with the drum 2, the rail 3 is forged from one part. The three free faces of the rail are induction hardened (58 (0/+4) HRC). The drum 2 is mounted in the frame 1 such that the annular rail 3 and the frame 1 substantially lie in one plane. Here, on the external side (i.e. the side facing the frame 1), the annular rail 3 is borne at three points on lower rollers 4 and an upper roller 5. These lower and upper rollers 4, 5 are either manufactured completely from through-hardened (59 (0/+4) HRC) ball-bearing steel (100Cr6 pursuant to DIN EN 10027-1) or else they have bearing surfaces made of through-hardened (59 (0/+4) HRC) ball-bearing steel (100Cr6 pursuant to DIN EN 10027-1). The lower rollers 4 and the upper roller 5 are mounted on the frame 1. In a polar coordinate system, in which the angular coordinate starts at 0 degrees at the top-most point in the opening of the frame 1 and then rotates in the plane of the frame 1 in a clockwise direction, respectively one pair of the lower rollers 4 is mounted on the frame 1 at 135° and at 225°. Since the frame 1 stands vertically, the weight of the drum 2 is borne by these two pairs of lower rollers 4. Both pairs of the lower rollers 4 are mounted on a rocker 6 in a fitting recess in the opening of the frame 1 with the axles of the lower rollers 4 being parallel to the axis of rotational symmetry of the drum 2 (see FIG. 3; in the view of FIG. 1, these rockers 6 are situated on the rear side of the frame 1). The rockers 6 can rock about an axis that is parallel to the axis of rotational symmetry of the drum 2. The axis of the rockers 6 lies in the mirror-symmetric plane of the rockers 6, which is situated perpendicularly with respect to the connecting line between the two axles of the lower rollers 4. The rocking motion made possible by the rockers 6 ensures that the weight of the drum 2 is uniformly distributed over all lower rollers 4.

The upper roller 5 is mounted eccentrically at 0° of the angular coordinate in the aforementioned polar coordinate system and clamps the drum 2 between itself and the lower rollers 4. Together, the upper and lower rollers 4, 5 prevent a displacement of the drum 2 in the plane of the frame 1 and at the same time permit a rotational movement of the drum 2 about the axis of rotational symmetry thereof.

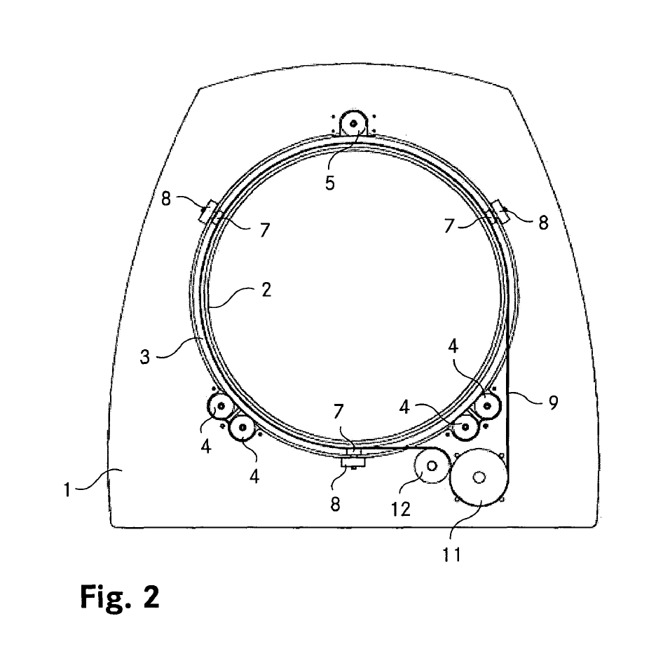

So that the rail 3 of the drum 2 and the frame 1 remain in one plane, guide rollers 7 are attached to the frame 1; these guide rollers consist of through-hardened (59 (0/+4) HRC) ball-bearing steel (100Cr6 pursuant to DIN EN 10027-1) or have at least bearing surfaces made of through-hardened ball-bearing steel (100Cr6 pursuant to DIN EN 10027-1). These guide rollers 7 are respectively arranged in pairs, wherein, per pair, one guide roller 7 is attached on one side of the face of the frame 1 (and the rail 3) and the other guide roller 7 is attached on the other side of the frame 1 (and the rail 3) and runs on the respective side face of the rail 3. Overall, three pairs of guide rollers 7 are arranged symmetrically around the drum 2 at angular coordinates 45°, 180° and 315° (see FIG. 2). The axles of the guide rollers 7 point radially away from the axis of rotational symmetry of the drum 2. On the side of the guide rollers 7 facing away from the axis of rotational symmetry of the drum 2, the axles of the guide rollers are held by guide-roller holders 8. These guide-roller holders 8 are fixedly mounted on the frame 1. The guide rollers 7 are mounted eccentrically on at least one side of the frame 1, as a result of which the drum 2 is mounted without play in the frame 1.

Arranged on the side of the frame 1 lying opposite to the rockers 6 there is a V-belt 9, which runs in a groove worked into the drum 2. The V-belt 9 is driven by a drive roller 11. It encircles the drum 2 and the drive roller 11 without crossing. The lower part of the V-belt 9 is pushed up, and thus tensioned, directly after the drive roller 11 by means of a tension roller 12. Both the drive roller 11 and the tension roller 12 are mounted on the frame 1. The axle of the drive roller passes through the frame 1 and is driven by a motor 13 on the other side of the frame 1.

On the side of the V-belt 9 facing away from the frame 1, a slip ring 14 has been attached to the drum 2 in an interface provided therefor, said slip ring establishing the electrical contacts for the instruments and components that are attached to the drum 2 (not illustrated) via a brush (not illustrated) attached to the frame 1. This contact to the slip ring 14 moreover enables precise positioning of the drum 2. Appropriate slip ring systems are known and are offered by e.g. Schleifring, Fürstenfeldbruck, Germany.

FIG. 2 shows a frontal view of the support system, according to the invention, for scanners. Here, the symmetric arrangement of the lower rollers 4 at angular coordinates 135° and 225° and the upper roller 5 at angular coordinate 0°, and also of the guide rollers 7 and the guide-roller holder 8 at angular coordinates 45°, 180° and 315° around the drum 2 are clearly visible.

FIG. 3 shows an oblique view of the support system, according to the invention, for scanners. In contrast to FIGS. 1 and 2, this view shows the rear side of the support system.

The two rockers 6 are mounted on the frame 1 and each hold two lower rollers 4. (The lower rollers 4 are only visible from the other side; see FIGS. 1 and 2.) A housing 15 is attached to the frame 1 and houses the eccentrically mounted upper roller 5. (The upper roller 5 is only visible from the other side; see FIGS. 1 and 2.)

A hole pattern (not illustrated) is situated on the side of the drum 2 shown here and enables the instruments and components (not illustrated) to be attached to the drum 2.

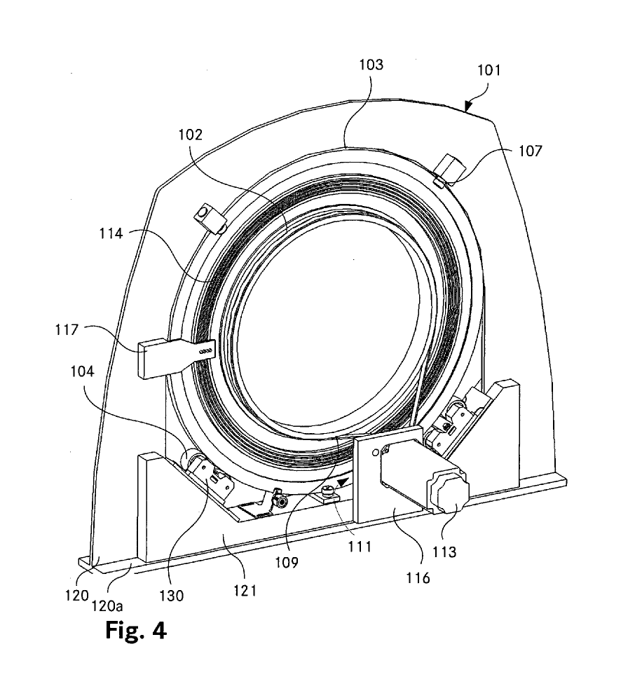

FIG. 4 is an oblique view of a second support system, according to the invention, for a scanner.

The illustrated support system once again comprises a frame 101, which is formed from a steel plate 120 on which, on the base side, a support plate 120a that runs perpendicular to said plate is arranged.

The frame 101 forms an opening, in which a substantially hollow-cylindrical drum 102 has space, with the axis of rotational symmetry thereof being perpendicular with respect to the plane of the frame 101. The diameter of this drum 102 is approximately eight times greater than the length of the drum 102. The drum 102 is forged without joints from heated and tempered steel C45 (pursuant to DIN EN 10027-1). A rail 103 with a rectangular cross section, which runs in the center of the lateral face of the drum 102, encompasses the drum 102 in an annular manner. Together with the drum 102, the rail 103 is forged from one part. The three free faces of the rail are induction hardened (58 (0/+4) HRC). The drum 102 is mounted in the frame 101 such that the annular rail 103 runs substantially in a central plane of the frame 101. Here, on the external side (i.e. the side facing the frame 101) the annular rail 103 is borne by four rollers 104 lying therebelow, which are arranged in two pairs and mounted on the frame 101 on a support structure 121 attached to the frame 101 on both sides. The rollers 104 are either manufactured completely from through-hardened (59 (0/+4) HRC) ball-bearing steel (100Cr6 pursuant to DIN EN 10027-1) or else they have bearing surfaces made of through-hardened (59 (0/+4) HRC) ball-bearing steel (100Cr6 pursuant to DIN EN 10027-1).

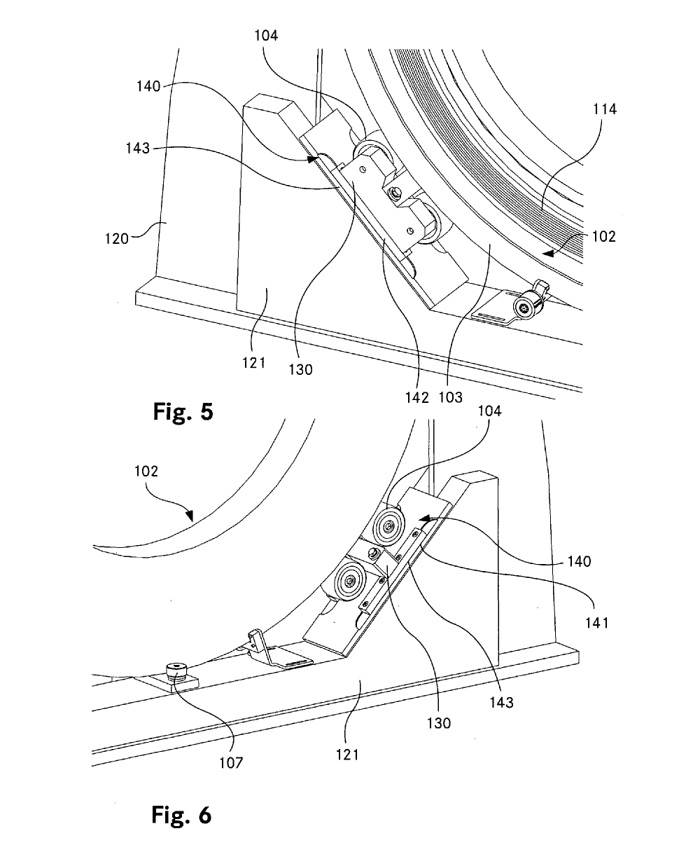

In a polar coordinate system, in which the angular coordinate starts at 0 degrees at the top-most point in the opening of the frame 101 and then rotates in the plane of the frame 101 in a clockwise direction, respectively one pair of the lower rollers 104 is mounted on the frame 101 at 135° and at 225°. The weight of the drum 102 is borne by these two pairs of lower rollers 104. Both pairs of the lower rollers 104 are rotatably attached to a roller support 130 with the axles of the lower rollers 104 being parallel to the axis of rotational symmetry of the drum 102. The roller support 130 can move, tangentially with respect to the drum 120, on a linear guide 140. It is described in detail below, in conjunction with FIGS. 5-8.

So that the rail 103 of the drum 102 and the frame 101 remain in one plane, guide rollers 107 are attached to the frame 101; these guide rollers consist of through-hardened (59 (0/−1-4) HRC) ball-bearing steel (100Cr6 pursuant to DIN EN 10027-1) or have at least bearing surfaces made of through-hardened ball-bearing steel (100Cr6 pursuant to DIN EN 10027-1). These guide rollers 107 are respectively arranged in pairs, wherein, per pair, one guide roller 107 is attached on one side of the frame 101 and hence the rail 103 and the other guide roller 107 is attached on the other side of the frame 101 and hence the rail 103, and run on the respective side face of the rail 103. The mounts of the roller axles are attached to the steel plate 120, the axles run radially inward. Overall, three pairs of guide rollers 107 are arranged symmetrically around the drum 102 at the angular coordinates 45°, 180° and 315°. The guide rollers 107 are mounted eccentrically on at least one side of the frame 101, as a result of which the drum 102 is mounted without play in the frame 101. The bearing surfaces for the rollers 104 formed on the rail 103 and the guide rollers 107 have been lubricated in order to reduce the noise development and the wear on the rollers.

Arranged on one side of the frame 101 there is a V-belt 109, which runs in a groove worked into the drum 102. The V-belt 109 is driven by a drive roller 111. It encircles the drum 102 and the drive roller 111 without crossing. The lower part of the V-belt 109 is pushed up, and thus tensioned, directly after the drive roller 111 by means of a tension roller (not illustrated) as known per se. The axle of the drive roller 111 is driven by a motor 113. The motor 113, the drive roller 111 and the tension roller 112 are mounted on a holding plate 116, which is attached to the support structure 121 on the outside, as seen axially.

A slip ring 114 has been attached to one side of the drum 102 in an interface provided therefor, said slip ring establishing the electrical contacts for the instruments and components that are attached to the drum 102 (not illustrated) via a brush 117 attached to the frame 101. This contact to the slip ring 114 moreover enables precise positioning of the drum 102. Appropriate slip ring systems are known and are offered by e.g. Schleifring, Fürstenfeldbruck, Germany.

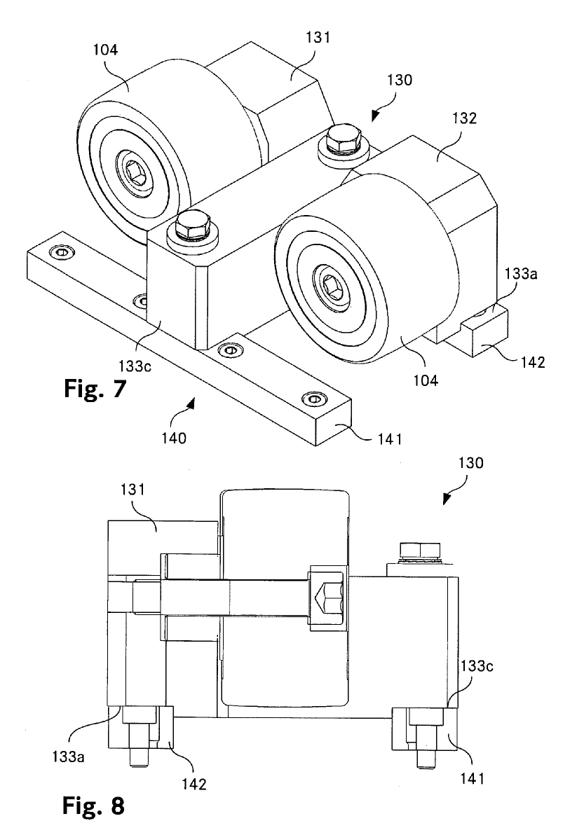

FIG. 5 is a detailed view, from a first side, of an installed roller support with two rollers. FIG. 6 is a detailed view of the roller support from a second side. FIG. 7 is an oblique view of the roller support and FIG. 8 shows a cross section through the roller support.

The roller support 130 comprises two roller bearings 131, 132, in which respectively one roller 104 is mounted on one side. The mounting is in this case brought about by means of pretensioned angular ball bearings, as can be seen in e.g. EP 1 907 717 B1 (Güdel Group AG). The two roller bearings 131, 132 are housed in a common housing 133. A groove 133a is formed on the underside of the housing 133. A central piece 133b of the housing extends forward, parallel to the axles of the rollers 104, with the front end of the central piece 133b projecting beyond the rollers 104. A further groove 133c is formed on the underside of the front end that projects beyond the rollers. Bearing surfaces are formed in both grooves 133a, 133c, and so the roller support 130 can slide on two rails 141, 142 of a linear guide 140. Applied to the contact areas of the rails 141, 142 there advantageously is a damping plastic layer, e.g. made of ethylene chlorotrifluoroethylene (ECTFE), which is commercially available under the trademark Turcite®.

The linear guide 140 comprises a base profile 143 to which the rails 141, 142 are screwed. In the outward direction, the base profile 143 extends upward at an angle of 45° with respect to the horizontal and is attached to the support structure 121 on both sides.

The movement made possible by the linear guide 140 ensures that the weight of the drum 102 is distributed uniformly on all lower rollers 104. Within the scope of operating the device, a path of approximately 5 mm is sufficient for the roller support 130.

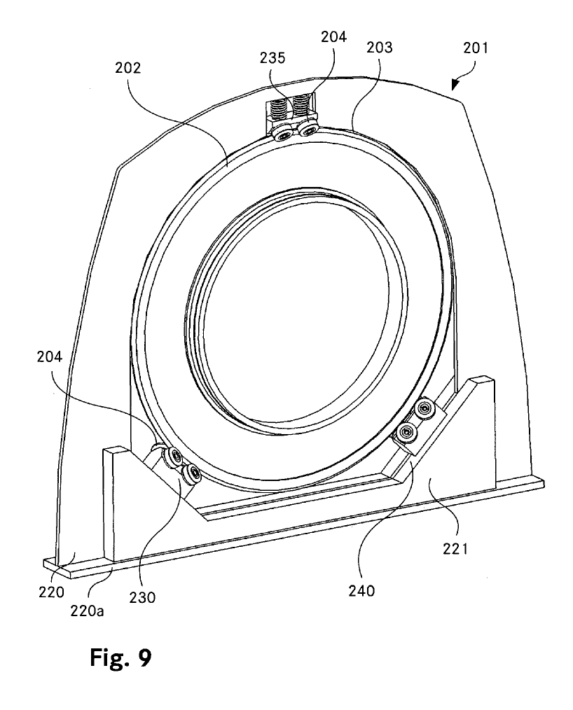

FIG. 9 shows an oblique view of a third support system, according to the invention, for a scanner. In many respects, the illustrated support system corresponds to that according to the second embodiment illustrated in FIGS. 4-8; the components with an identical design, such as the drive or the means for data transmission from the drum to the frame, have therefore no longer been illustrated.

The illustrated support system once again comprises a frame 201, which is formed from a steel plate 220, on which, on the base side, a support plate 220a that runs perpendicular to said plate is arranged. The frame 201 forms an opening, in which a substantially hollow-cylindrical drum 202 has space, with the axis of rotational symmetry thereof being perpendicular with respect to the plane of the frame 201. The diameter of this drum 202 is approximately eight times greater than the length of the drum 202. The drum 202 is forged without joints from heated and tempered steel C45 (pursuant to DIN EN 10027-1). On the outside, the drum comprises a raceway 203 with a prism-shaped cross section, on which two bearing surfaces are formed which are respectively arranged at 45° to the rotational axis of the drum and hence include an angle of 90°. The bearing surfaces of the raceway 203 are induction hardened (58(0/+4) HRC). The drum 202 is mounted in the frame 201 such that the edge at which the two bearing surfaces meet runs substantially in a central plane of the frame 201. Here, on the external side (i.e. the side facing the frame 201) the raceway 203 is borne by eight rollers 204 lying therebelow. Four of these rollers 204 are respectively rotatably mounted on a roller support 230, with each of the two roller supports 230 respectively having two rollers 204 on each side of the central plane of the frame 201. The rotary axles of the rollers are angled at 45° with respect to the rotational axis of the drum 202 and arranged such that they interact with the bearing surface of the respective side, i.e. two roller pairs are opposite to one another with rotary axles that are angled at 90° with respect to one another.

The roller supports 230 are mounted on the frame 201 on a support structure 221 attached to the frame 201 on both sides. The rollers 204 are either manufactured completely from through-hardened (59 (0/+4) HRC) ball-bearing steel (100Cr6 pursuant to DIN EN 10027-1) or else they have bearing surfaces made of through-hardened (59 (0/+4) HRC) ball-bearing steel (100Cr6 pursuant to DIN EN 10027-1). The outer surfaces of the rollers are ground like a ball such that differential slip is prevented in the region of the running contact.

In a polar coordinate system, in which the angular coordinate starts at 0 degrees at the top-most point in the opening of the frame 201 and then rotates in the plane of the frame 201 in a clockwise direction, respectively one pair of the lower rollers 204 is mounted on the frame 201 at 135° and at 225°. The weight of the drum 202 is borne by these two pairs of lower rollers 204. The roller support 230 can move, tangentially with respect to the drum 220, on a linear guide 240. It is described in detail below, in conjunction with FIG. 11.

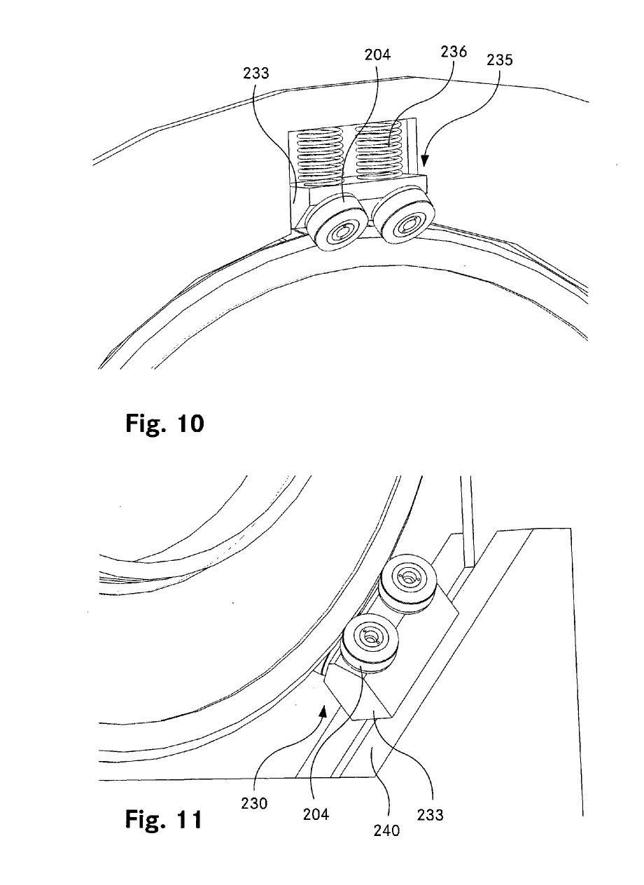

A third roller support 235 with four further rollers 204 is arranged at the top, at 0°. This roller support 235 also interacts with both bearing surfaces of the raceway 203. It is described in detail in conjunction with FIG. 10.

The rollers 204 assume the supporting function (in the case of the lower roller supports 230) and at the same time laterally guide the drum 202 as a result of their angled arrangement, i.e. hold it in the plane of the opening of the frame 201.

The bearing surfaces for the rollers 204 formed on the raceway 203 are lubricated in order to reduce the noise development and the wear on the rollers.

FIG. 10 is a detailed view of an upper roller support 235 and FIG. 11 is a detailed view of a lower roller support 230, respectively in the installed state.

The roller supports 230, 235 respectively comprise four roller bearings, in which respectively one roller 204 is mounted on one side. The mounting is in this case preferably brought about by means of pretensioned angular ball bearings, as can be seen in e.g. EP 1 907 717 B1 (Güdel Group AG). The four roller bearings are housed in a common housing 233.

Analogously to the roller supports as per the second embodiment, the lower roller supports 230 are also linearly moveable along a guide 240 running tangentially with respect to the drum 202. Applied to the contact face between the guide 240 and the roller support 230 there advantageously is a damping plastic layer, e.g. made of ethylene chlorotrifluoroethylene (ECTFE), which is commercially available under the trademark Turcite®. The movement made possible by the linear guide 240 ensures that the weight of the drum 202 is distributed uniformly on all lower rollers 204. Within the scope of operating the device, a path of approximately 5 mm is sufficient for the roller support 230.

The upper roller support 235 is mounted on the frame 201 via springs 236. The springs 236 carry the roller support 235 and hence the rollers 204 onto the drum 202 and thereby ensure continuous contact between the bearing surfaces and the rollers 204. As a result, diameter variations as a result of thermal influences can be absorbed.

The invention is not restricted to the illustrated exemplary embodiments. Depending on the specific application, the edge of the drum between the instruments and components attached to the drum can for example have recesses, as a result of which the overall mass of the drum is reduced. This enables the use of a motor designed for less power and an accordingly less strong V-belt.

Moreover, the V-belt can for example by replaced by a toothed belt or other types of driving belts.

By way of example, depending on the specific application, it is also possible to vary the arrangement of the driving belt, the slip ring and the holes for attaching instruments and components on the drum. In doing so, driving belts, slip ring and also holes can be placed on the drum on one side of the frame or the other.

Depending on the application of the support system, it is possible to modify the arrangement and number of rollers. Accordingly, the positions of the guide rollers can also be varied as required.

In conclusion, it should be noted that the invention creates a support system for scanners, which enables short examination times but nevertheless has a cost-effective design.

1. A support system for a scanner, comprising a frame and a drum, wherein mounting of the drum on the frame allows a rotation of the drum about the axis of rotational symmetry thereof and wherein a scanner system can be attached to the drum, characterized in that the drum substantially consists of heated and tempered steel and/or has bearing surfaces made of hardened steel and in that a roller system with rollers is attached to the frame, by means of which roller system the drum can be rotated about the axis of rotational symmetry of the drum, wherein the rollers consist of hardened steel or are at least equipped with bearing surfaces made of hardened steel.

2. The support system as claimed in claim 1, characterized in that the drum is produced without joints, more particularly it is forged or rolled.

3. The support system as claimed in claim 1, characterized in that, on the lateral face, the drum comprises a rail on which the rollers of the roller system run.

4. The support system as claimed in claim 1, characterized in that the weight of the drum is borne by at least two roller pairs, which are each arranged moveably relative to the frame along a track oriented tangentially with respect to the drum.

5. The support system as claimed in claim 4, characterized in that at least two rollers are attached to a roller support in each case, wherein the roller support is mounted on a linear guide attached to the frame and wherein a damping element is preferably arranged between the roller support and the linear guide.

6. The support system as claimed in claim 1, characterized in that the weight of the drum is borne by a plurality of roller pairs, which are each mounted on a rocker.

7. The support system as claimed in claim 1, characterized in that one or more further rollers which do not bear the weight of the drum are mounted eccentrically or are under resilient pretension.

8. The support system as claimed in claim 1, characterized in that the drum is held in the frame by guide rollers attached to the frame by virtue of the guide rollers running on both sides of the rail and by virtue of the axles of the guide rollers being aligned radially with respect to the drum, wherein the guide rollers consist entirely of hardened steel or are equipped with at least one bearing surface made of hardened steel.

9. The support system as claimed in claim 8, characterized in that the drum is mounted without play by virtue of the guide rollers on at least one side of the rail (3; 103) being mounted eccentrically or being under resilient pretension.

10. The support system as claimed in claim 1, characterized in that the rotational movement of the drum is driven by a driving belt that runs around the drum.

11. The support system as claimed in claim 1, characterized in that the motor for the driving belt is attached to the frame.

12. The support system as claimed in claim 1, characterized in that the driving belt is tensioned by means of a tension roller.

13. The support system as claimed in claim 1, characterized in that a slip ring on the external side of the drum establishes the required electrical contacts to the instruments that are attached to the drum.

14. The support system as claimed in claim 1, characterized in that as a result of a contact to the slip ring on the external side of the drum, the precise positioning of the drum is ensured.

15. The support system as claimed in claim 1, characterized in that the radiation source and the detector or detectors are attached to holes in the drum.

16. The support system as claimed in claim 2, characterized in that, on the lateral face, the drum comprises a rail on which the rollers of the roller system run.

17. The support system as claimed in claim 2, characterized in that the weight of the drum is borne by at least two roller pairs, which are each arranged moveably relative to the frame along a track oriented tangentially with respect to the drum.

18. The support system as claimed in claim 3, characterized in that the weight of the drum is borne by at least two roller pairs, which are each arranged moveably relative to the frame along a track oriented tangentially with respect to the drum.

19. The support system as claimed in claim 2, characterized in that the weight of the drum is borne by a plurality of roller pairs, which are each mounted on a rocker.

20. The support system as claimed in claim 3, characterized in that the weight of the drum is borne by a plurality of roller pairs, which are each mounted on a rocker.
